(19) United States
(12) Patent Application Publication
(10) Pub. No.: US 2018/0021476 A1
(43) Pub. Date: Jan. 25, 2018

(54) MEDICAL IMPLANT

(71) Applicant: INVIBIO LIMITED, Lancashire (GB)

(72) Inventor: Adam BRISCOE, Thornton Cleveleys (GB)

(73) Assignee: INVIBIO LIMITED, Lancashire (GB)

(21) Appl. No.: 15/536,436

(22) PCT Filed: Dec. 16, 2015
(86) PCT No.: PCT/GB2015/054025

§ 371 Date: Jun. 15, 2017

(30) Foreign Application Priority Data
Dec. 18, 2014 (GB) 1422559.3

(51) Int. Cl. A61L 27/18; A61L 27/50; C08J 7/12; A61L 27/26

(52) U.S. Cl. CPC A61L 27/18; A61L 27/26; A61L 27/50; C08J 7/123; A61L 2430/02; C08J 2371/08; A61F 2/3662

(57) ABSTRACT

The invention relates to an orthopaedic implant comprising a surface arranged to contact bone in a human or animal body. The surface of said implant comprises a polymeric material which includes a repeat unit of general formula

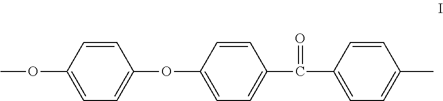

The polymeric material is polyetheretherketone, and said surface is a plasma treated surface.

This invention relates to a medical implant and particularly, although not exclusively, relates to an orthopaedic implant comprising a polymeric material, for example polyetheretherketone (PEEK).

Medical implants, such as orthopaedic implants, which comprise polymeric materials, such as PEEK, have been proposed. For example, US2010/0312348A (Howmedica) discloses an orthopaedic polyaryletherketone (PAEK) on polymer bearing and proposes a PEEK-based femoral head. However, the document does not describe how the implants may be secured in a femur.

It is known to use polymethylmethacrylate (PMMA) in surgery (see for example J. Bone Joint Surg. Br JULY 2007 vol 89-B no.7 851-857). For example, during a hip replacement, the PMMA may be arranged in the femur and a femoral component of the hip implant may be positioned in the PMMA. Although PMMA is often referred to as a cement, it is more properly referred to as a grout which forms a micro-interlock between interstices in cancellous bone and in grooves formed in the femoral component of the implant itself. However, an interlock alone between the femoral component and PMMA may be insufficient in some cases.

It is known that PMMA is a very poor adhesive in general and is especially poor if it is attempted to use it as an adhesive for PEEK. It is an object of the preferred embodiment of the present invention to address this problem.

In general terms, it is an object of preferred embodiments of the invention to enhance securement or a medical implant within an opening defined in a bone. Especially preferred embodiments aim to enhance securement of a component of a medical implant within a femur during a hip or knee replacement.

According to a first aspect of the invention, there is provided an orthopaedic implant comprising a surface arranged to contact bone in a human or animal body, wherein said surface of said implant comprises a polymeric material which includes a repeat unit of general formula

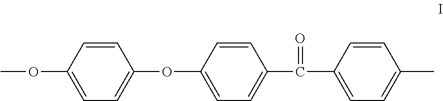

wherein said surface is a plasma treated surface.

Said polymeric material may include at least 80 wt %, preferably at least 90 wt %, more preferably at least 95 wt %, especially at least 99 wt % of repeat units of formula I.

Said polymeric material suitably includes at least 90 mol %, preferably at least 95 mol %, more preferably at least 99 mol % of repeat units of formula I.

Said polymeric material preferably consists essentially of a repeat unit of formula I. Preferably, said polymeric material is polyetheretherketone.

Said polymeric material may have a Notched Izod Impact Strength (specimen 80 mm×10 mm×4 mm with a cut 0.25 mm notch (Type A), tested at 23° C., in accordance with ISO180) of at least 4 KJm⁻², preferably at least 5 KJm⁻², more preferably at least 6 KJm⁻². Said Notched Izod Impact Strength, measured as aforesaid, may be less than 10 KJm⁻², suitably less than 8 KJm⁻². The Notched Izod Impact Strength, measured as aforesaid, may be at least 3 KJm⁻², suitably at least 4 KJm⁻², preferably at least 5 KJm^{31 2}. Said impact strength may be less than 50 KJm⁻², suitably less than 30 KJm⁻².

Said polymeric material suitably has a melt viscosity (MV) of at least 0.06 kNsm⁻², preferably has a MV of at least 0.09 kNsm⁻², more preferably at least 0.12 KNsm⁻², especially at least 0.15 kNsm⁻². Advantageously, the MV may be at least 0.35 kNsm⁻² and especially at least 0.40 kNsm⁻². An MV of 0.45 kNsm⁻² has been found to be particularly advantageous.

MV is suitably measured using capillary rheometry operating at 400° C. at a shear rate of 1000 s⁻¹ using a circular cross-section tungsten carbide die, 0.5 mm (capillary diameter)×3.175 mm (capillary length).

Said polymeric material may have a MV of less than 1.00 kNsm⁻², preferably less than 0.5 kNsm⁻².

Said polymeric material may have a MV in the range 0.09 to 0.5 kNsm⁻², preferably in the range 0.14 to 0.5 kNsm⁻², more preferably in the range 0.4 to 0.5 kNsm⁻².

Said polymeric material may have a tensile strength, measured in accordance with ISO527 (specimen type 1b) tested at 23° C. at a rate of 50 mm/minute of at least 20 MPa, preferably at least 60 MPa, more preferably at least 80 MPa. The tensile strength is preferably in the range 80-110 MPa, more preferably in the range 80-100 MPa.

Said polymeric material may have a flexural strength, measured in accordance with ISO178 (80 mm×10 mm×4 mm specimen, tested in three-point-bend at 23° C. at a rate of 2 mm/minute) of at least 50 MPa, preferably at least 100 MPa, more preferably at least 145 MPa. The flexural strength is preferably in the range 145-180 MPa, more preferably in the range 145-164 MPa.

Said polymeric material may have a flexural modulus, measured in accordance with ISO178 (80 mm×10 mm×4 mm specimen, tested in three-point-bend at 23° C. at a rate of 2 mm/minute) of at least 1 GPa, suitably at least 2 GPa, preferably at least 3 GPa, more preferably at least 3.5 GPa. The flexural modulus is preferably in the range 3.5-4.5 GPa, more preferably in the range 3.5-4.1 GPa.

Said polymeric material may be amorphous or semi-crystalline. It is preferably crystallisable. It is preferably semi-crystalline.

The level and extent of crystallinity in a polymer is preferably measured by wide angle X-ray diffraction (also referred to as Wide Angle X-ray Scattering or WAXS), for example as described by Blundell and Osborn (Polymer 24, 953, 1983). Alternatively, crystalling may be assessed by Differential Scanning Calorimetry (DSC).

The level of crystalling of said polymeric material may be at least 1%, suitably at least 3%, preferably at least 5% and more preferably at least 10%. In especially preferred embodiments, the crystallinity may be greater than 25%. It may be less than 50% or less than 40%.

The main peak of the melting endotherm (Tm) of said polymeric material (if crystalline) may be at least 300° C.

The fact a surface of said polymeric material has been plasma treated as described may be confirmed by use of ESCA—for example the surface of the polymeric material may have an increase in the number of oxygen groups. In addition, the contact angle with water at the surface may be reduced compared to an equivalent surface which differs only in it not having been plasma treated as described.

Said orthopaedic implant may have a first surface area which represents the entire surface area of the implant. Suitably, less than 100%, preferably less than 90%, more preferably less than 80%, for example less than 70%, of the first surface area is plasma treated as aforesaid. At least 40%, of said first surface area may be plasma treated. In a preferred embodiment, only a back side of the implant which contacts the cement in use is treated.

Said first surface area may be at least 3500 mm². Said first surface may be less than to 25000 mm².

Said surface arranged to contact bone may have an Ra in at least one region thereof of at least 5 μm, preferably at least 10 μm, more preferably at least 100 μm. Said Ra preferably extends across at least 50% or at least 80% of the area of said surface. Said Ra may extend across at least 20%, suitably at least 50%, preferably at least 70% of said first surface area described.

Said orthopaedic implant suitably includes at least 50 wt %, preferably at least 75 wt%, more preferably at least 85 wt %, especially at least 95 wt % of said polymeric material. In the most preferred embodiment said orthopaedic implant comprises at least 99 wt % of said polymeric material.

Said orthopaedic implant is preferably arranged to cooperate with a bone associated with a joint, for example a hip, knee, shoulder, elbow, wrist or ankle. In a preferred embodiment, said implant is arranged to cooperate with a femur. Said implant may comprise a femoral stem, for a hip arthroplasty or may comprise a knee component for a knee arthroplasty. Said orthopaedic implant may be arranged to cooperate with a socket, opening or recess defined in or by a bone.

Said implant, for example said femoral stem or knee component, may be arranged to cooperate with a second orthopaedic member thereby to define an assembly for use in arthroplasty, for example in hip or knee arthroplasty. Thus, said orthopaedic implant of the first aspect may be a component of an assembly or arrangement (e.g., comprising a selected orthopaedic implant and a selected second orthopaedic member, wherein the implant and member are arranged to be assembled together). Said assembly or arrangement as described may include said orthopaedic implant and said second orthopaedic member. Said second orthopaedic member may comprise a metal, ceramic or polymeric material. Said second orthopaedic member preferably comprises a polymeric material. Said second orthopaedic member may comprise a polyolefin. Said second orthopaedic member may comprise a polyethylene, a polyurethane, a polyacetal or a polyamide. In a preferred embodiment, said second orthopaedic member comprises a polyolefin, for example a polyethylene such as UHMWPE. It may comprise a cross-linked UHMWPE. Said second orthopaedic member suitably includes at least 50 wt %, preferably at least 75 wt %, more preferably at least 85 wt %, especially at least 95 wt % of said polymeric material as described. In the most preferred embodiment, said second orthopaedic member comprises at least 99 wt % of said polymeric material.

In some cases, part of said second orthopaedic member may be plasma treated as described herein.

The invention extends, in a second aspect, to an arrangement comprising:
(i) an orthopaedic implant as described in the first aspect; (i) a second orthopaedic member arranged to be assembled with the orthopaedic implant to define an orthopaedic assembly for arthroplasty, for example, hip, knee, shoulder, elbow, wrist or ankle arthroplasty.

The implant described in (i) and the member described in (ii) may be spaced apart in the arrangement by a distance of less than 5 m, for example less than 1 m.

The arrangement of the second aspect may also comprise a cement or one or more precursors arranged to form such a cement, for securing the orthopaedic implant to a bone. The cement (or at least one precursor thereof) is suitably acrylic-based. Said one or more precursors is suitably arranged to define an acrylic-based cement, for example an acrylate cement, preferably a polymeric cement. Said precursor may be arranged to define a polyacrylate cement, for example a polymethylmethacrylate (PMMA) cement. Thus, said arrangement of the second aspect may comprise a polyacrylate, for example a PMMA cement or one or more precursors arranged to produce such a cement.

The cement or said one or more precursors may, in the arrangement, be spaced from the orthopaedic implant described in (i) by a distance of less than 5 m, for example less than 1 m.

The invention extends, in a third aspect, to an assembly comprising:
(i) an orthopaedic implant as described in the first aspect;
(ii) a second orthopaedic member assembled with the orthopaedic implant to define an orthopaedic assembly for use in arthroplasty.

The orthopaedic implant is suitably associated with, for example secured in position, by a cement which is suitably acrylic-based. Said cement may be an acrylate cement. Said cement is preferably a polymeric cement. Said cement is preferably a poyacrylate cement, for example a PMMA cement.

In the assembly, said cement preferably contacts said plasma treated surface of said orthopaedic implant. In the assembly, preferably, the only surface of said orthopaedic implant which said cement contacts is a surface of said orthopaedic implant which is a plasma treated surface. Thus, said cement preferably does not contact a surface of said orthopaedic implant which is not a plasma treated surface.

According to a fourth aspect of the invention, there is provided a method of preparing for surgery, for example arthroplasty, the method comprising: (a) selecting an orthopaedic implant according to the first aspect, (b) selecting a cement for securing the implant to a bone or selecting one or more precursors arranged to define a cement.

The cement (or at least one precursor thereof) is suitably acrylic-based. Said one or more precursors is suitably arranged to define an acrylic-based cement, for example an acrylate cement, preferably a polymeric cement. Said precursor may be arranged to define a polyacrylate cement, for example a polymethylmethacrylate (PMMA) cement.

The method may comprise a step (c) which comprises selecting a second orthopaedic member (suitably being as described in the preceding aspects) which arranged to cooperate with said orthopaedic implant referred to in (a) to define an assembly for use in arthroplasty, for example in hip, knee, shoulder, elbow, wrist or ankle arthroplasty. Steps (a), (b) and/or (c) may be in any order. The method may include a step (d) which comprises selecting a tool for treating a bone to define a surface to which said orthopaedic implant referred to in (a) can be secured.

In one embodiment, said method may comprise selecting a tool for defining a socket in a bone in which said orthopaedic implant may be secured.

According to a fifth aspect of the invention, there is provided a method of securing an orthopaedic implant too a bone of a human or animal body, the method comprising:
(A) selecting an orthopaedic implant according to the first aspect;
(B) cementing the implant to a bone of the human or animal body, suitably using an acrylic-based cement, for example an acrylate cement. Said cement is preferably a polymeric cement. Said cement is preferably a polyacrylate cement, for example a PMMA cement.

The method suitably comprises positioning the orthopaedic implant relative to the bone so said plasma treated surface is contacted with said cement in step (B).

The method may comprise positioning the orthopaedic implant in a socket, for example defined in a femur, so that said plasma treated surface is within the socket and is contacted with said cement in step (B).

The method may comprise selecting a precursor of said cement and causing said precursor to react to define said cement in situ.

According to a sixth aspect of the invention, there is provided a combination comprising:
(i) an orthopaedic implant as described in the first aspect; and
(ii) an acrylic based cement;
   said combination being for use in arthroplasty, for example in hip, knee, shoulder, elbow, wrist or ankle arthroplasty.

The combination may be for securing the orthopaedic implant to a bone of the human or animal body. Suitably, the combination is for positioning in a socket of a bone (for example the femur), wherein said implant extends within said socket and said cement is arranged to cement the implant in position in said socket, suitably so the implant is substantially immovably arranged within said socket.

The implants and/or methods referred to in any aspect herein are preferably for use in arthroplasties, for example hip, knee, shoulder, elbow, wrist or ankle arthroplasties.

Preferred treatments herein are of the human body.

Any feature of any aspect of the invention or embodiment described herein may be combined with any other feature of any aspect of an invention or embodiment described herein mutatis mutandis.

Specific embodiments of the invention will now be described, by way of example, with reference to the accompanying drawings, in which:

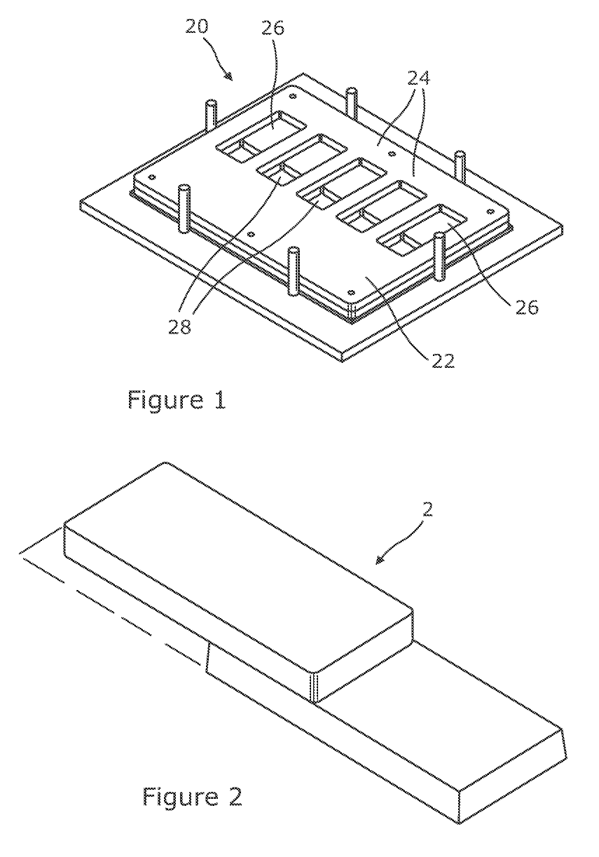

FIG. 1 is a perspective view of part of an apparatus used to prepare samples for assessment; and

FIG. 2 is a representation of a lap-joint.

The following material is referred to hereinafter:

PEEK-OPTIMA (Trade Mark)—refers to grade LTI polyetheretherketone (PEEK) having a melt viscosity of 0.45 KNsm⁻² obtained from Invibio Ltd. UK.

Various samples comprising PEEK-OPTIMA were made, with different roughnesses and surface treatments and were tested to assess how adhesion between PEEK and PMMA could be improved. Example 1 describes a general method of preparing a substrate; example 2 describes how substrates may be plasma treated; examples 3 to 20 describe specific samples; examples 21 and 22 describe how samples may be prepared and tested.

### Example 1—General Method for Preparing PEEK-OPTIMA Substrates

PEEK-OPTIMA substrates in the form of plates having dimensions 6 mm×25 mm×60 mm were prepared by injection moulding PEEK using standard conditions. In some cases, smooth PEEK-OPTIMA substrates were prepared; in other cases the PEEK-OPTIMA substrates were post-treated (e.g. by grit-blasting) to increase surface roughness (Ra).

### Example 2—Plasma Treated of PEEK-OPTIMA Substrate

Substrates were plasma treated using a commercially available Nano series plasma surface machine by Diener Electronics (Germany) having the following features:
Chamber—cylindrical with 24 litres capacity.
Plasma generator power used: 100%.
Plasma generator type: Radio frequency signal of 40 kHz.
Gas used: Oxygen 99% pure.
Admission of oxygen time: 5 minutes.
Plasma treatment time: 30 minutes.

### Example 3—Samples Prepared

Using the procedures described in Examples 1 and 2, a range of samples were made for testing as described in Table 1 below. Note that all plates made consist of PEEK-OPTIMA unless otherwise stated.

The Ra surface roughness value of samples was measured using a TALYSURF (Trade Mark) contact surface profilometer, with a standard diamond stylus, following ISO4288-1996 in each case, six measurements were taken across each sample in the direction of the tensile force to be applied during shear testing.

TABLE 1

| | | Surface |
|---|---|---|
| Example | | Roughness |
| No. | Description of Example | (Ra) in μm |
| 4 | Plasma treated as described in Example 2. | 0.5 |
| 5 | Post treated and plasma treated as described | 100* |
| | in Example 2. | |
| 6 | Post treated and plasma treated as described | 200* |
| | in Example 2. | |
| 7 | Post treated as Example 5 (but without | 100* |
| | plasma treatment). | |
| 8 | Post treated as Example 6 (but without | 200* |
| | plasma treatment). | |
| 9 | Grit blasted to specified roughness. | 6 |
| 10 | Grit blasted to specified roughness. | 3 |
| 11 | Grit blasted to specified roughness. | 6 |
| 12 | Post treated having the specified roughness. | 70* |
| 13 | Post treated having the specified roughness. | 20 |
| 14 | Post treated having the specified roughness. | 40 |
| 15 | Post treated having the specified roughness. | 17 |
| 16 | Post treated having the specified roughness. | 15 |
| 17 | Post treated having the specified roughness. | 20 |
| 18 | Post treated having the specified roughness. | 70* |
| 19 | Post treated having the specified roughness. | 300* |
| | A fine pitched rib. | |
| 20 | Post treated having the specified roughness. | 10* |
| | A stepped plaque. | |

*micro surface roughnss Ra-1 μm.

### Example 21—Preparation of Cemented Samples for Testing

A cementing mould assembly 20, shown in FIG. 1, includes a mould part 22, which includes five rectangular openings 24 in which samples 2, shown in FIG. 2, are prepared. To prepare samples for testing, the assembly 20 is set up with 25 mm×25 mm areas 28 of each sample exposed. Cement was mixed in accordance with the manufacturer's instructions. Then, using a powder-free gloved hand, cement was finger packed into each mould, ensuring each mould was fully filled. Then a polyethylene spacer sheet was positioned to cover the samples, a lid applied and G-clamps applied. The cement was accordingly cured under pressure in accordance with the manufacturer's instructions. After the appropriate time, the mould was disassembled and samples pressed out by hand. Samples were stored and conditioned for three days prior to testing as described in Example 22.

### Example 22—Testing of Interfacial Strength of Samples

Testing was performed on an Instron 5569 electromechanical test machine with a 50 kN load cell. The sample was positioned vertically in the jaws using 6 mm thickness aluminium alignment plates so that the load axis was coincident with the lap interface of the test sample, with an equal area of either end of the sample supported in the grips. Samples were then loaded at a rate of 2 mm/min to failure.

Shear interface strength was calculated from the following equation.

$$Shear strength ( MPa ) = \frac{failure load ( N )}{Lap joint area ( {mm}^{2} )}$$

Statistical analysis was then performed with IBM SPSS Statistics 20. Analysis of variance (ANOVA) tests with 5% significance criteria were chosen to allow multiple comparisons of mean strength between all independent sample groups tested (assuming normal distribution). Test results are provided in Table 2.

TABLE 2

| | Average shear |
|---|---|
| Example No. | strength, MPa |
| 4 | 0.37 |
| 5 | 3.16 |
| 6 | 2.39 |
| 7 | 1.24 |
| 8 | 1.12 |
| 9 | 1.53 |
| 10 | 0.62 |
| 11 | 1.31 |
| 12 | 0.77 |
| 13 | 0.91 |
| 14 | 1.18 |
| 15 | 0.52 |
| 16 | 0.51 |
| 17 | 1.35 |
| 18 | 0.94 |
| 19 | 2.07 |
| 20 | 0.52 |

Note that attempts were made to test untreated and untextured PEEK-OPTIMA samples (i.e. like Example 4 but without the plasma treatment) but the bond strength between the PEEK-OPTIMA and the cement was so low that the samples could not be removed from the mould so no relevant quantitative result could be obtained.

The results in Table 2 show, unpredictably, that the plasma treatment can be used to significantly increase the strength of the bond between PMMA and PEEK-OPTIMA. The bond is, in many cases, stronger than for samples which have post treated surface features (resulting in significant Ra) and significant mechanical interlock with the PMMA.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An orthopaedic implant comprising a surface arranged to contact bone in a human or animal body, wherein said surface of said implant comprises a polymeric material which includes a repeat unit of general formula 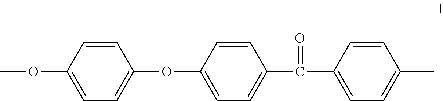
wherein said polymeric material is polyetheretherketone, and wherein said surface is a plasma treated surface.

2. An implant according to claim 1 wherein the level of crystallinity of said polymeric material is greater than 25%.

3. An implant according to claim 1, wherein said orthopaedic implant has a first surface area which represents the entire surface area of the implant, wherein less than 100% of the first surface area is plasma treated as aforesaid.

4. An implant according to claim 3, wherein at least 40% of said first surface area is plasma treated.

5. An implant according to claim 1, wherein a back side of the implant which contacts cement in use, is treated.

6. An implant according to claim 1, wherein said surface arranged to contact bone has an Ra in at least one region thereof of at least 5 μm.

7. An implant according to claim 6, wherein said Ra extends across at least 50% of the area of said surface.

8. An implant according to claim 1, wherein said implant comprises a femoral stem for a hip arthroplasty or a knee component for a knee arthroplasty.

9. An implant according to claim 1, wherein said implant is arranged to cooperate with a second orthopaedic member thereby to define an assembly for use in arthroplasty.

10. An implant according to claim 1, wherein said orthopaedic implant includes at least 50 wt % of said polymeric material.

11. An arrangement comprising:
(i) an orthopaedic implant as described in claim 1,
(ii) a second orthopaedic member arranged to be assembled with the orthopaedic implant to define an orthopaedic assembly.

12. An arrangement according to claim 11, wherein said second orthopaedic member comprises a polymeric material.

13. An arrangement according to claim 11, wherein the arrangement comprises a cement or one or more precursors arranged to form such a cement, for securing the orthopaedic implant in position.

14. An arrangement according to claim 13, wherein the cement, or at least one precursor of the cement, is acrylic-based.

15. An arrangement according to claim 14, wherein a precursor of said cement is arranged to define a polyacrylate cement.

16. An assembly comprising:
(i) an orthopaedic implant as described in claim 1;
(ii) a second orthopaedic member assembled with the orthopaedic implant to define an orthopaedic assembly.

17. An assembly according to claim 15, wherein the orthopaedic implant is associated with a cement which is acrylic-based.

18. An assembly according to claim 17, wherein said cement is a polyacrylate cement.

19. An assembly according to claim 17, wherein said cement contacts said plasma treated surface of said orthopaedic implant.

20. An assembly according to claim 16 wherein, in the assembly, the only surface of said orthopaedic implant which said cement contacts is a surface of said orthopaedic implant which is a plasma treated surface.

21. A method of preparing for surgery, the method comprising:
(a) selecting an orthopaedic implant according to claim 1;
(b) selecting a cement for securing the implant to bone or selecting one or more precursors arranged to define a cement.

22. A method according to claim 21, wherein the method comprises a step (c) which comprises selecting a second orthopaedic member which is arranged to cooperate with said orthopaedic implant referred to in (a) to define an assembly for use in arthroplasty.

23. A method of securing an orthopaedic implant to a bone of a human or animal body, the method comprising:
(A) selecting an orthopaedic implant according to claim 1;
(B) cementing the implant to a bone of the human or animal body.

24. A combination comprising:
(I) an orthopaedic implant as described in claim 1;
(II) an acrylic-based cement;
said combination being for use arthroplasty.
